(19) United States
(12) Patent Application Publication
(10) Pub. No.: US 2026/0083924 A1
(43) Pub. Date: Mar. 26, 2026

(54) CAPSULE INHALER FOR THE ADMINISTRATION OF A PHOSPHODIESTERASE-4 INHIBITOR

(71) Applicant: CHIESI FARMACEUTICI S.P.A., Parma (IT)

(72) Inventors: Francesca BUTTINI, Parma (IT); Giada VARACCA, Parma (IT); Romina OSELLO, Parma (IT)

(73) Assignee: CHIESI FARMACEUTICI S.P.A., Parma (IT)

(21) Appl. No.: 19/112,743

(22) PCT Filed: Sep. 21, 2023
(86) PCT No.: PCT/EP2023/076014

§ 371 Date: Mar. 18, 2025

(30) Foreign Application Priority Data
Sep. 22, 2022 (EP) 22197239.1

(51) Int. Cl. A61M 15/00; A61K 9/00; A61K 31/4425; A61M 15/08

(52) U.S. Cl. CPC A61M 15/0021; A61K 9/0075; A61K 31/4425; A61M 15/0035; A61M 15/08; A61M 2202/064

(57) ABSTRACT

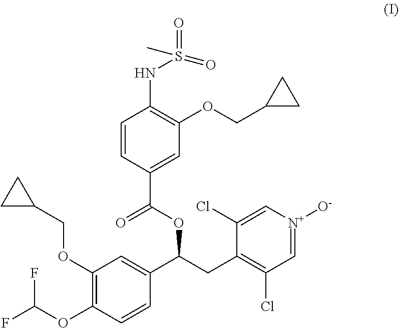 [SMILES: CS(=O)(=O)Nc1ccc(C(=O)O[C@@H](Cc2c(Cl)c[n+]([O-])cc2Cl)c2ccc(OC(F)F)c(OCC3CC3)c2)cc1OCC1CC1]

The present invention relates to a drug product comprising a single-dose dry powder inhalation device and a pharmaceutical composition loaded in a capsule, the pharmaceutical composition comprising micronized particles of the compound of formula (I) and a carrier. The present invention also relates to a pharmaceutical composition for use for the treatment of a respiratory disease and to a method for the treatment of a respiratory disease.

### FIELD OF THE INVENTION

The present invention relates to a drug product comprising a single-dose dry powder inhalation device and a pharmaceutical composition loaded in a capsule, the pharmaceutical composition comprising micronized particles of the compound of formula (I) and a carrier. The present invention also relates to a pharmaceutical composition for use for the treatment of a respiratory disease and to a method for the treatment of a respiratory disease.

### BACKGROUND OF THE INVENTION

The compound of formula (I)

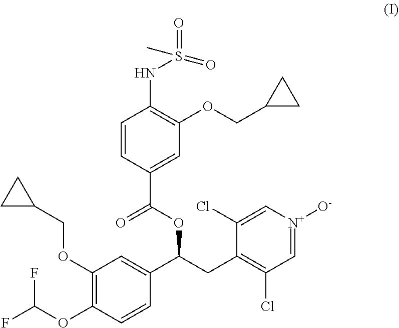 [SMILES: CS(=O)(=O)Nc1ccc(C(=O)O[C@@H](Cc2c(Cl)c[n+]([O-])cc2Cl)c2ccc(OC(F)F)c(OCC3CC3)c2)cc1OCC1CC1]

also named tanimilast or CHF6001 or CHF-6001, with INN (3,5-dichloro-4-[(2S)-2-[3-(cyclopropylmethoxy)-4-(difluoromethoxy)phenyl]-2-{[3-(cyclopropylmethoxy)-4-(methanesulfonamido)benzoyl]oxy}ethyl]pyridinel-oxide), is an highly potent and selective PDE4 inhibitor with robust anti-inflammatory activity, currently under clinical development.

Compound of formula (I) has been disclosed in prior art documents in the name of Chiesi: WO 2009/018909 directed to its general formula, methods of preparation, compositions and therapeutic use; WO 2010/089107 specifically directed to sulphonamido derivatives as (−) enantiomers, including compound of formula (I), methods of preparation, compositions and therapeutic use; WO 2012/016889 directed to dry powder compositions comprising the compound of formula (I); WO 2015/059050 directed to crystal form, named Form A, of the compound of formula (I) characterized by specific XRPD peaks and the process for obtaining it.

As other members of the pharmacological class of PDE4 inhibitors, said drug may be indicated for the treatment of lung diseases such as asthma, chronic obstructive pulmonary disease (COPD), bronchiectasis, chronic bronchitis, pneumonia, acute respiratory distress syndrome (ARDS), pulmonary emphysema, smoking-induced emphysema and cystic fibrosis.

Due to well-known systemic side effects associated to the class of PDE4 inhibitors, tanimilast is under development as a composition for inhalation.

In fact, one of the advantages of the inhalatory route over the systemic one is the possibility of delivering the drug locally at site of action, significantly avoiding any systemic absorption and related side effects.

Currently, tanimilast is in an advanced clinical stage in the form of a powder composition exploiting the platform technology disclosed in WO 2012/016889 and is administered through the proprietary multidose Nexthaler® inhaler. Said product is indicated hereinafter as the “Reference Product”.

As a carrier, a fissured coarse lactose and a fraction constituted of a mixture of fine lactose and magnesium stearate as a ternary agent are used. Said composition, as disclosed in WO 2012/016889, is indicated hereinafter as the “Reference Composition”.

Thanks to the property of both the inhaler and the platform technology, the composition provides an excellent respirable fraction as well as a significant amount of extrafine particles.

Tanimilast is under investigation at two single doses per actuation, 400 μg and 800 μg.

Although systemic effects of inhaled drugs are not a serious concern because absorption is low, assuming that the 800 μg dose would turn out to be the most effective, there could be an increase of the risk of adverse events associated to systemic absorption.

Furthermore, for drugs to be administered as powder composition, a dose of 800 μg, corresponding to a concentration of 4%, would not be optimal from a manufacturing point of view. In fact, agglomerates could form, affecting the homogeneity of the active ingredient in the blend. A non optimal homogeneity in turn could increase the risk of an over or under dosage.

Thus, it would be advantageous to provide a platform technology for the compound of formula (I) which could provide better inhalatory performances, so allowing administering the drug at a lower dose, while retaining the therapeutic effect of the higher dose. Said platform technology shall also give rise to the same fraction of extrafine particles.

There is indeed consensus about the fact that extrafine particles are capable of reaching the distal tract of the respiratory tree and hence improving small airways outcomes and associated control in the patients affected by the small airways asthma phenotype (Santus P, Respir Care 2020; 65 (9): 1392-1412; Scichilone N, Patient Relat Outcome Meas 2014; 5:153-162.).

The technical solution is provided by the present invention.

### SUMMARY OF THE INVENTION

In a first aspect, the present invention is directed to a drug product comprising a single-dose dry powder inhalation device, comprising an inhaler body (2) defining a recess (3) for a capsule (4), wherein the capsule (4) holds herein a pharmaceutical composition to be inhaled, a nosepiece or mouthpiece (5) communicating with the recess (3), at least one rupturing element (7) coupled to the inhaler body (2) and configured for rupturing the capsule (4) to allow an outside airflow to be mixed with the pharmaceutical composition of the capsule (4) and inhaled through the nosepiece or the mouthpiece (5), and a pharmaceutical composition filled in a capsule, the pharmaceutical composition comprising micronized particles having a size comprised between 0.1 and 15 micron of a compound of formula (I) and carrier, particles, wherein the inspiratory flow rate of said inhalation device is between 30 l/min and 65 l/min at 4 kPa of pressure drop, and wherein the unitary nominal dose of the compound of formula (I) is comprised between 450 and 600 μg.

Advantageously the carrier comprises a ternary agent.

Preferably, the carrier is constituted of:
a) a fraction of fine particles made of a mixture composed of 90 to 99.5 percent by weight of particles of a physiologically acceptable excipient and 0.5 to 10 percent by weight of a ternary agent, said mixture having a volume median diameter lower than 20 micron; and
b) a fraction of coarse particles constituted of a physiologically acceptable excipient having a volume median diameter equal to or higher than 100 micron, as measured by means of laser diffraction or sieve analyzer, wherein the ratio between the fine particles and the coarse particles being between 1:99 and 30:70 percent by weight.

In a second aspect, the invention is directed to a pharmaceutical composition according to the invention for use for the treatment of a respiratory disease, wherein said composition is administered using a single-dose dry powder inhalation device whose inspiratory flow rate is comprised between 30 l/min and 65 l/min at 4 kPa of pressure drop, and wherein the unitary nominal dose of the compound of formula (I) is comprised between 450 and 600 μg.

In a third aspect, the invention provides a method for the treatment of a respiratory disease, wherein the method comprises administering the compound of formula (I) by inhalation to a patient, wherein the drug product is as described according to the invention and wherein the nominal dose of the compound of formula (I) per actuation is comprised between 450 and 600 μg.

In a fourth aspect, the invention provides a process for the preparation of a drug product according to the invention, said process comprising the step of:
a) preparing microparticles constituted of a mixture composed of particles made of physiologically acceptable pharmacologically-inert material and particles of the additive, the inert material and the additive being first-mixed together and then co-micronized;
b) mixing the microparticles of step a) with coarse particles of a physiologically acceptable pharmacologically-inert material such that microparticles adhere to the surface of the coarse particles;
c) adding by mixing the active particles in the micronized form to the particles of step b) to obtain the final pharmaceutical composition;
d) filling the obtained final pharmaceutical composition in a capsule; and
e) loading the medicament chamber of the single dry powder inhalation device with the capsule.

### BRIEF DESCRIPTION OF THE FIGURES

FIG. 1: comparative deposition in the Next Generation Impactor of 20 mg of 800 microg formulations having the composition of the Reference Product aerosolized by Nexthaler, HR RS01 or UHR RS01 device

FIG. 2: in vitro dissolution profile of the Reference Product at 400 and 800 μg dose

FIG. 3: comparative in vitro dissolution of the Reference Product and the drug product of the invention at 800 μg dose upon aerosolization by Nexthaler, HR RS01 or UHR RS01

FIG. 4: three-dimensional view of a single-dose dry powder inhalation device according to an embodiment of the present invention

FIG. 5: cross section of the single-dose dry powder inhalation device of FIG. 4 in a first operational configuration

FIG. 6: cross section of the single-dose dry powder inhalation device of FIG. 4 in a second operational configuration

### DETAILED DESCRIPTION OF THE INVENTION

### Definitions

Unless defined otherwise, all technical and scientific terms used herein have the same meanings as commonly understood by the skilled in the art.

Unless otherwise specified, the compound of formula (I) of the present invention is intended to include also polymorphs, stereoisomers, tautomers or pharmaceutically acceptable salts or solvates thereof.

The term “micron”, “micrometers” and μm are used as synonymous.

The term “microgram” and μg are used as synonymous.

The term “percent” and % are used as synonymous.

Unless otherwise specified, the compound of formula (I) of the present invention is intended to include also polymorphs, stereoisomers, tautomers or pharmaceutically acceptable salts or solvates thereof.

The term “pharmaceutically acceptable salts”, as used herein, refers to derivatives of compounds of formula (I) wherein the parent compound is suitably modified by converting any of the free acid or basic group, if present, into the corresponding addition salt with any base or acid conventionally intended as being pharmaceutically acceptable. Suitable examples of said salts may thus include mineral or organic acid addition salts of basic residues such as amino groups, as well as mineral or organic basic addition salts of acid residues such as carboxylic groups.

Cations of inorganic bases which can be suitably used to prepare salts comprise ions of alkali or alkaline earth metals such as potassium, sodium, calcium or magnesium.

Those obtained by reacting the main compound, functioning as a base, with an inorganic or organic acid to form a salt comprise, for example, salts of hydrochloric acid, hydrobromic acid, sulfuric acid, phosphoric acid, methane sulfonic acid, camphor sulfonic acid, acetic acid, oxalic acid, maleic acid, fumaric acid, succinic acid and citric acid.

The term “solvate” means a physical association of a compound of this invention with one or more solvent molecules, whether organic or inorganic. This physical association includes hydrogen bonding. In certain instances, the solvate will be capable of isolation, for example, when one or more solvent molecules are incorporated in the crystal lattice of the crystalline solid. The solvate may comprise either a stoichiometric or nonstoichiometric amount of the solvent molecules.

The term “stereoisomer” refers to isomers of identical constitution that differ in the arrangement of their atoms in space. Enantiomers and diastereomers are examples of stereoisomers.

The term “racemate” or “racemic mixture” refers to a composition composed of equimolar quantities of two enantiomeric species, wherein the composition is devoid of optical activity.

The term “tautomer” refers to each of two or more isomers of a compound that exist together in equilibrium and are readily interchanged by migration of an atom or group within the molecule.

The term “composition”, as in pharmaceutical composition, is intended to encompass a product comprising the active ingredient and any pharmaceutically acceptable excipient or carrier, as well as any product which results, directly or indirectly, from combination, complexation or aggregation of any two or more of the ingredients, or from dissociation of one or more of the ingredients, or from other types of reactions or interactions of one or more of the ingredients.

By the term “physiologically acceptable” it is meant a safe, pharmacologically-inert substance utilized as an excipient.

The term “vitro-in vivo correlation” (IVIVC) refers to an in vitro dissolution test that is predictive of the in vivo performance of the drug product.

By the term “micronized” it is meant a substance having a size of few microns, typically comprised between 0.1 and 15 micron.

By the term “fine particles” it is meant particles having a size up to few tenths of microns.

By the term “extrafine particles” it is meant particles having a particle size equal or less than 2.0 micron.

The term “coarse” refers to a substance having a size of one or few hundred microns.

The term “surface coating” refers to the covering of the surface of the excipient particles by forming a thin film of the ternary agent around said particles.

The terms “additive” and ternary agent” are used as synonymous, and with this term, we mean substances that could modify the detachment of the active ingredient from the surface of the carrier particles, increasing the respirable fraction.

In general terms, the particle size of particles is quantified by measuring a characteristic equivalent sphere diameter, known as volume diameter, by laser diffraction or sieve analyzer.

The particle size can also be quantified by measuring the mass diameter by means of suitable known instrument such as, for instance, the sieve analyser or laser diffraction.

The volume diameter (VD) is related to the mass diameter (MD) by the density of the particles (assuming a size independent density for the particles).

In the present application, the particle size of the active ingredients and of fraction of fine particles is expressed in terms of volume diameter.

The particles have a log-normal distribution which is defined in terms of the volume or mass median diameter (VMD or MMD) which corresponds to the volume or mass diameter of 50 percent by weight of the particles, and, optionally, in terms of volume or mass diameter of 10% and 90% of the particles, respectively.

Another common approach to define the particle size distribution is to cite three values: i) the median diameter d(0.5), which is the diameter where 50% of the distribution is above and 50% is below; ii) d(0.9), where 90% of the distribution is below this value; iii) d(0.1), where 10% of the distribution is below this value. If said diameter is determined as equivalent volume diameter (the diameter of the hypothetical sphere having the same volume as the particle under examination), the three parameters are indicated as dv(0.5), dv(0.9) and dv(0.1).

VMD corresponds to dv(0.5). MMD corresponds to d(0.5).

The span is the width of the distribution based on the 10%, 50% and 90% quantile and is calculated according to the formula.

$$Span = \frac{D [ v , 0.9 ] - D [ v , 0.1 ]}{D [ v , 0.5 ]}$$

In general terms, particles having the same or a similar VMD or MMD can have a different particle size distribution, and in particular a different width of the Gaussian distribution, as represented by the d(0.1) and d(0.9) values.

Upon aerosolisation, the particle size is expressed as mass aerodynamic diameter (MAD), while the particle size distribution is expressed in terms of mass median aerodynamic diameter (MMAD) and Geometric Standard Deviation (GSD). The MAD indicates the capability of the particles of being transported suspended in an air stream. The MMAD corresponds to the mass aerodynamic diameter of 50 percent by weight of the particles.

The term “hard pellets” refers to spherical or semispherical units whose core is made of coarse excipient particles.

The expression “respirable fraction” refers to an index of the percentage of active particles which would reach the lungs in a patient. The respirable fraction, also indicated as Fine Particle Fraction, (FPF), is evaluated using a suitable in vitro apparatus such as Andersen Cascade Impactor (ACI), Multi Stage Liquid Impinger (MLSI) or Next Generation Impactor (NGI), according to procedures reported in common Pharmacopoeias, in particular in the European Pharmacopeia (Eur. Ph.) 11 Edition, paragraph 2.9.18, 372-378. It is calculated by the percentage ratio of the fine particle mass (FPM) (formerly fine particle dose, FPD) to the delivered dose.

The term “peak inspiratory flow rate” refers to the maximal rate of the flow of air during inspiration of the patient through or without the inhalation device.

The term “inspiration flow rate” refers to the constant rate of the flow of air capable to generate a pressure drop across the inhaler of 4.0 kPa (40.8 cm H₂O) during in vitro test in accordance to the European Pharmacopeia (Eur. Ph.) 11 Edition, paragraph 0671 Preparations for Inhalation: Inhalanda, 998.

The delivered dose is calculated from the cumulative deposition in the
apparatus, while the fine particle mass is calculated from the deposition of particles having a diameter equal or lower than 5.0 micron.

In the context of the present application, the composition is defined as “extrafine” composition when it is able of delivering a fraction of particles having a particle size equal or less than 2.0 micron equal to or higher than 20%, preferably equal to or higher than 25%, more preferably equal to or higher than 30% and/or it is able of delivering a fraction of particles having a particle size equal or less than 1.0 micron equal to or higher than 10%.

The expression “physically stable in the device before use” refers to a composition wherein the active particles do not substantially segregate and/or detach from the surface of the carrier particles both during manufacturing of the dry powder and in the delivery device before use. The tendency to segregate can be evaluated according to Staniforth et al. J. Pharm. Pharmacol. 34,700-706, 1982 and it is considered acceptable if the distribution of the active ingredient in the powder composition after the test, expressed as relative standard deviation (RSD), does not change significantly with respect to that of the composition before the test.

The term “prevention” means the slowing of the progression, delaying the onset, and/or reducing the risk of contracting the disease.

The term “treatment” means an approach for obtaining beneficial or desired results, including clinical results. Beneficial or desired clinical results can include, but are not limited to, alleviation or amelioration of one or more symptoms or conditions, diminishment of extent of disease, stabilized (i. e. not worsening) state of disease, preventing spread of disease, delay or slowing of disease progression, amelioration or palliation of the disease state, and remission (whether partial or total), whether detectable or undetectable. The term can also mean prolonging survival as compared to expected survival if not receiving treatment.

“Unitary therapeutically unitary effective dose” or “unitary nominal dose” means the quantity of active ingredient to be administered at one time by inhalation upon actuation of the inhaler. Said dose may be delivered in one or more actuations of the inhalation device, preferably one or two actuations (shot) of the device, more preferably one actuation of the device.

“Actuation” refers to the release of active ingredients from the device by a single activation (e.g. mechanical or breath).

“Daily dose” means the quantity of active ingredient to be administered in a day by inhalation upon actuation of the inhaler.

The term “delivered dose” refers to the amount of drug effectively delivered to the respiratory tree after each actuation of the inhaler.

The expression “good homogeneity” refers to a composition wherein, upon mixing, the uniformity of distribution of the active ingredient, expressed as coefficient of variation (CV) also known as relative standard deviation (RSD), is less than 5%, preferably less than 2.5%, more preferably equal to or less than 1.5%.

The pharmaceutical compositions of the invention comprehend any type of composition made by admixing the compound of the invention and pharmaceutically acceptable excipients and/or carriers.

On the basis of the required inspiratory flow rates (l/min) which in turn are strictly depending on their design and mechanical features, DPI's are also divided in:
i) low-resistance devices (about 100 l/min);
ii) medium-resistance devices (about 80 l/min);
iii) high-resistance devices (about 65 l/min);
iv) ultra-high resistance devices (about 40 l/min).

The reported flow rates refer to the pressure drop of 4 kPa (KiloPascal) in accordance to the European Pharmacopeia (Eur. Ph.) 11 Edition, paragraph 0671 Preparations for Inhalation: Inhalanda, 998.

It has been found that, by using a single-dose dry powder inhalation device with an inspiratory flow rate comprised between 30 l/min and 65 l/min at 4 kPa of pressure drop in combination with a high-performance powder technology platform, it is possible to improve the inhalatory performances, in particular the FPF, so allowing administering the drug at a lower dose, while retaining the therapeutic effect of the higher dose.

In fact, the results reported in Tables 5 and 7 of Examples 3 of the experimental part indicate that, using a single-dose dry powder inhalation device with an inspiratory flow rate comprised between 30 l/min and 65 l/min at 4 kPa of pressure drop in combination with the same technology platform of the Reference Composition, an increment of about 25-35% of the FPF could be achieved in comparison to the Reference Product.

Also the extraFPF turned out to have an increment of about 28-58% with respect to the Reference Product.

The increase is calculated by the ratio of the difference between the value of the test product and that of the reference by the value of the reference and multiplied by 100.

The improved performances are confirmed by the in vitro dissolution results using a system specifically set up to assess the in vitro dissolution profiles of drug upon inhalation.

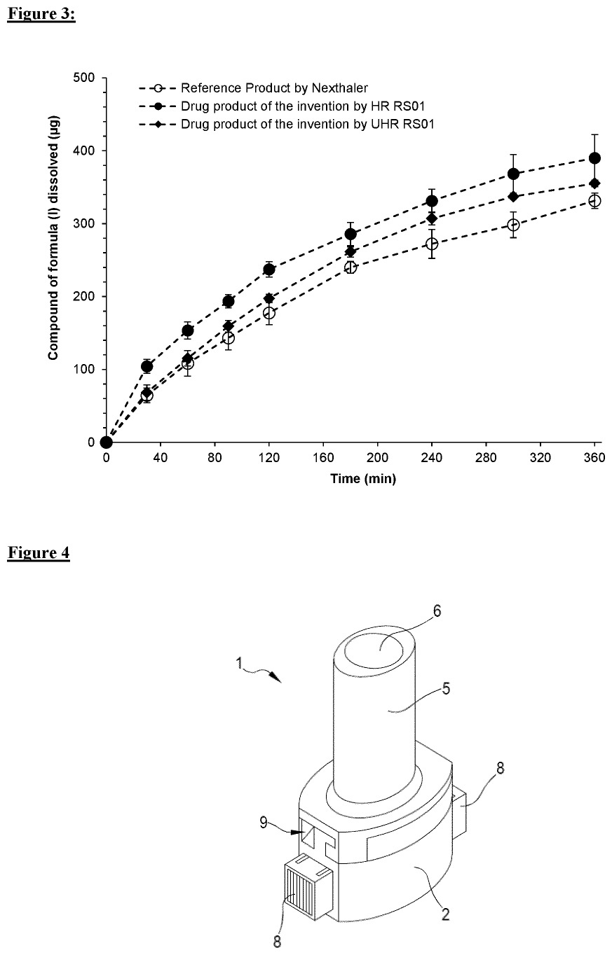

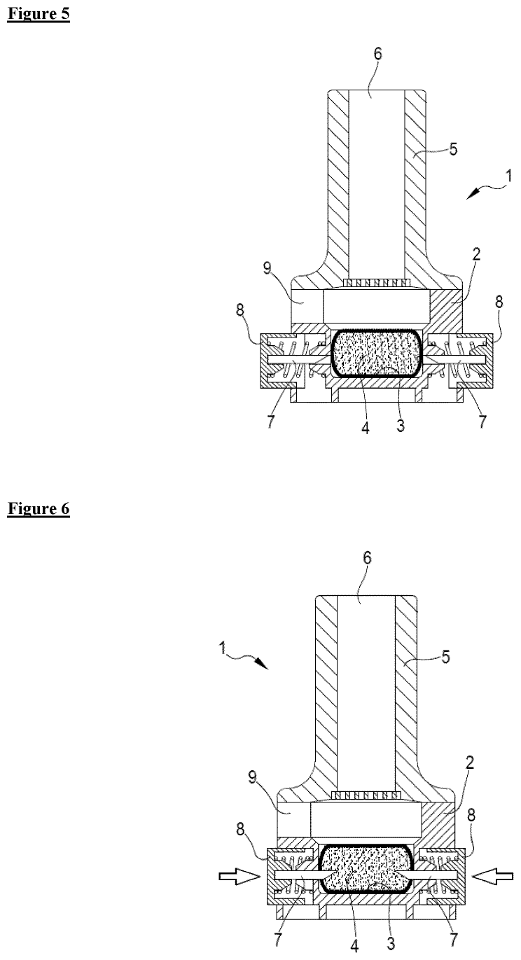

As it can be appreciated from FIG. 3, an amount of the compound of formula (I) of about 7-20% higher was dissolved in comparison to the Reference Product. This would allow to reduce the 800 μg unitary nominal dose of about 25-35%. In the art, capsule-based single-dose dry-powder inhaler were utilized to administer tanimilast. For example, in Mariotti F., International Journal of COPD, 2018:13 3399-3410, it is described a first study, wherein tanimilast was administered via a the capsule-based single-dose dry-powder inhaler Aerolizer, and in the second study, the same compound was administered via a reservoir-based multi-dose dry-powder inhaler (MDDPI) NEXThaler®. According to the results, the weighted AUC systemic bioavailability of the compound of formula (I) of the present invention was about 30% higher following administration via the multi-dose dry-powder inhaler Nexthaler than via the single-dose dry-powder inhaler Aerolizer, suggesting that, contrary to what was found in the present invention, the Nexthaler may provide better pulmonary drug deposition. Aerolizer is a low resistance device (0.019 kPa^{1/2}/L*min-1) it means that a inspiratory flow rate of about 105 l/min is required to achieve a 4 kPa pressure drop (Dal Negro, R. W. Dry powder inhalers and the right things to remember: a concept review. Multidiscip Resp Med 10, 13 (2015)).

Therefore, in a preferred embodiment of the present invention, with reference to the attached figures, the single-dose dry powder inhalation, which has been generally indicated by the reference number 1, comprises an inhaler body 2 defining a recess 3 for a capsule 4 and a nosepiece or mouthpiece 5 which communicates with the recess 3 and has an opening 6. Two rupturing elements 7 are coupled to the inhaler body 2 and are configured for rupturing the capsule 4 to allow an outside airflow to be mixed with a pharmaceutical composition contained in the capsule 4 and inhaled through the nosepiece or the mouthpiece 5. The two rupturing elements 7 of the single-dose dry powder inhalation device 1 of this embodiment are shaped like pegs or needles and are configured to perforate the capsule 4 when buttons 8 carrying the rupturing elements 7 are pushed and the capsule 4 is located in the recess 3. Air inlets 9 are provided in the inhaler body 2. Said air inlets 9 communicates with the recess 3 to allow the airflow to enter the recess 3 when the user inhales through the nosepiece or mouthpiece 5. A shape and size of the cited air inlets 9 may determine the intrinsic resistance to airflow of the single-dose dry powder inhalation device.

In a preferred embodiment, the present invention provides a drug product comprising a single-dose dry powder inhalation device selected from high-resistance and an ultra-high resistance devices. More preferably, the high resistance device is RS01 with code 239700002AA and the ultra-high resistance device RS01 with code 239700005AA.

The inspiratory flow rate is comprised between 30 l/min and 65 l/min as referred to the pressure drop of 4 kPa, preferably between 35 l/min and 65 l/min, more preferably between 40 l/min and 65 l/min, even more preferably between 35 l/min and 55 l/min, even more preferably is 65 l/min, even more preferably is 40 l/min.

The unitary nominal dose to be delivered after each actuation of the inhaler shall be comprised between 450 μg and 600 μg, preferably between 480 μg and 550 μg.

The daily dose at which the pharmaceutical composition comprising the compound of general formula (I) shall be administered is comprised between 800 μg and 4800 μg, preferably between 1200 μg and 3800 μg and more preferably between 1600 μg and 3200 μg.

In one embodiment the daily dose may be reached by a single or double administration.

In another preferred embodiment, the daily dose may be reached by a single administration and delivered in one actuation of the inhaler.

In another preferred embodiment the daily dose may be reached by a single administration and delivered in more actuations of the inhaler, preferably two.

In another preferred embodiment the daily dose may be reached by a double administration and delivered in one actuation of the inhaler.

In another preferred embodiment the daily dose may be reached by a double administration and delivered in more actuations of the inhaler, preferably two.

Advantageously, the carrier comprises a mixture of fine and coarse excipient particles constituted of any physiologically acceptable material or combination thereof, suitable for inhalatory use.

For example, said particles may be constituted of one or more materials selected from polyols, for example sorbitol, mannitol and xylitol, and crystalline sugars, including monosaccharides and disaccharides; inorganic salts such as sodium chloride and calcium carbonate; organic salts such as sodium lactate; and other organic compounds such as urea, polysaccharides, for example starch and its derivatives; oligosaccharides, for example cyclodextrins and dextrins.

Preferably, said particles are made of a crystalline sugar, even more preferably selected from: a monosaccharide such as glucose or arabinose, or a disaccharide such as maltose, saccharose, dextrose or lactose.

Preferably, said particles are made of lactose, more preferably of alpha-lactose monohydrate as said excipient is chemically and physically stable upon storage and easy to handle.

Preferably, the coarse excipient particles and the fine excipient particles are both constituted of alpha-lactose monohydrate.

The fraction of fine particles a) must have a mass median diameter (MMD) lower than 20 micron, advantageously equal to or lower than 15 micron, preferably equal to or lower than 10 micron, even more preferably equal to or lower than 6 micron.

Advantageously, the mass diameter of 90% of the fine particles a) is lower than 35 micron, more advantageously lower than 25 micron, preferably lower than 15 micron, even more preferably lower than 10 micron.

The ratio between the excipient particles and the ternary agent within the fraction a) may vary depending on the doses of the active ingredients.

Advantageously, said fraction is composed of 90 to 99.5% by weight of the excipient and 0.5 to 10% by weight of the ternary agent, preferably of 95 to 99% of the excipient, and 1 to 5% of the ternary agent. A preferred ratio is 98% of the excipient and 2% of the ternary agent.

Said ternary agent may be an amino acid, preferably selected from the group consisting of leucine, isoleucine, lysine, valine, methionine, and phenylalanine.

Alternatively, the ternary agent may include or consist of one or more water-soluble surface-active materials, for example lecithin.

In certain embodiments, the ternary agent may include or consist of one or more lubricant selected from the group consisting of stearic acid and salts thereof such as magnesium stearate, sodium lauryl sulphate, sodium stearyl fumarate, stearyl alcohol, sucrose monopalmitate.

Said ternary agent may be an amino acid, preferably selected from the group consisting of leucine, isoleucine, lysine, valine, methionine, and phenylalanine.

Alternatively, the ternary agent may include or consist of one or more water-soluble surface-active materials, for example lecithin.

In certain embodiments, the ternary agent may include or consist of one or more lubricant selected from the group consisting of stearic acid and salts thereof such as magnesium stearate, sodium lauryl sulphate, sodium stearyl fumarate, stearyl alcohol, sucrose monopalmitate. The preferred active material is magnesium stearate.

When magnesium stearate is used as ternary agent, depending on e.g. its amount and the time of mixing, magnesium stearate may coat the surface of the fine excipient particles in such a way as that the extent of the molecular surface coating is at least of 5%, preferably more than 10%, more preferably more than 15%, even more preferably equal to or more than and 25%.

The extent of molecular surface coating, which indicates the percentage of the total surface of the excipient particles coated by magnesium stearate, may be determined by water contact angle measurement, as reported in literature, for instance in WO 2011/120779.

The fraction of fine particles a) may be prepared according to one of the methods disclosed in WO 01/78693. Preferably, it could be prepared by co-micronization, more preferably using a ball mill. In some cases, co-milling for at least two hours may be found advantageous, although it will be appreciated that the time of treatment will generally depend on the starting particle size of the excipient particles and the desired size reduction to be obtained.

In a preferred embodiment of the invention the particles are co-micronized starting from excipient particles having a mass diameter less than 250 micron and magnesium stearate particles having a mass diameter less than 35 micron using a jet mill, preferably in inert atmosphere, for example under nitrogen.

As an example, alpha-lactose monohydrate commercially available such as Meggle D 30 or Spherolac 100 (Meggle, Wasserburg, Germany) could be used as starting excipient.

Optionally, the fraction of fine particles a) may be subjected to a conditioning step according to the conditions disclosed in the pending application n. WO 2011/131663

The coarse excipient particles of the fraction b) must have an MMD of at least 100 micron, preferably greater than 125 micron, more preferably equal to or greater than 150 micron, even more preferably equal to or greater than 175 micron.

Advantageously, all the coarse particles have a mass diameter in the range comprised between 50 and 1000 micron, preferably comprised between 60 and 500 micron.

In certain embodiments of the invention, the mass diameter of said coarse particles might be comprised between 80 and 200 micron, preferably between 90 and 150 micron, while in another embodiment, the mass diameter might be comprised between 200 and 400 micron, preferably between 210 and 355 micron.

In a preferred embodiment of the invention, the mass diameter of the coarse particles is comprised between 210 and 355 micron.

In general, the person skilled in the art shall select the most proper size of the coarse excipient particles by sieving, using a proper classifier.

When the mass diameter of the coarse particles is comprised between 200 and 400 micron, the coarse excipient particles preferably have a relatively highly fissured surface, that is, on which there are clefts and valleys and other recessed regions, referred to herein collectively as fissures. The “relatively highly fissured” coarse particles can be defined in terms of fissure index or rugosity coefficient as described in WO 01/78695 and WO 01/78693, incorporated herein by reference, and they can be characterized according to the description therein reported. Said coarse particles may also be characterized in terms of tapped density or total intrusion volume measured as reported in WO 01/78695, whose teaching is incorporated herein by reference.

The tapped density of said coarse particles is advantageously less than 0.8 g/cm³, preferably between 0.8 and 0.5 g/cm³. The total intrusion volume is of at least 0.8 cm³ preferably at least 0.9 cm³.

The ratio between the fraction of fine particles a) and the fraction of coarse particles b) is comprised between 1:99 and 30:70% by weight, preferably between 2:98 and 20:80% by weight. In a preferred embodiment, the ratio is comprised between 10:90 and 15:85% by weight, even more preferably is of 10:90% by weight.

The step of mixing the coarse excipient particles b) and the fine particles a) is typically carried out in a suitable mixer, e.g. tumbler mixers such as Turbula™, rotary mixers or instant mixer such as Diosna™ for at least 5 minutes, preferably for at least 30 minutes, more preferably for at least two hours. In a general way, the person skilled in the art shall adjust the time of mixing and the speed of rotation of the mixer to obtain a homogenous mixture.

When spheronized coarse excipient particles are desired in order to obtain hard-pellets according to the definition reported above, the step of mixing shall be typically carried out for at least four hours.

In a preferred embodiment, the carrier of the composition according to the invention is constituted of:
a) a fraction of fine particles made of a mixture composed of 98 percent by weight of particles of alpha-lactose monohydrate and 2 percent by weight of magnesium stearate, said mixture having a mass median diameter equal to or lower than 6 micron; b) a fraction of coarse particles constituted of alpha-lactose monohydrate having a mass diameter comprised between 210 and 355 micron and the ratio between the fine particles and the coarse particles being 10:90 percent by weight.

More preferably the physiologically acceptable excipient used as a coarse carrier has the d(0.1) comprised between 170 and 190 micron, the d(0.5) comprised between 270 and 300 micron and the d(0.9) comprised between 300 and 400 micron, all the values are expressed as mass diameter.

As it is explained above, the diameter of the particles measured by volume diameter by suitable tools such as laser diffraction or sieve analyzer, could be converted in the equivalent mass diameter knowing the density of the particles.

Advantageously, the compound of formula (I) has the following distribution measured as equivalent volume diameter: dv(0.1) comprised between 0.5 and 1 micron, the dv(0.5) comprised between 1.9 and 2.5 micron, the dv(0.9) comprised between 4 and 6 micron, with the span comprised between 1.7 and 2.3 micron.

In fact, span values in this range ensure that the population distribution of microparticles is distributed around the diameter median value. Therefore for small values of dv(0.5) (<2.5 μm), there will be in parallel a high portion of extra-fine particles which will favor a peripheral deposition of the drug in the lungs.

The particle size of the compound of formula (I) may be measured by laser diffraction as a dispersion, e.g., using a Mastersizer instrument (Malvern instruments). In particular, the technique is wet dispersion. The equipment is set with the following optical parameters: Refractive index for compound of formula (I)=1.52, Refractive index for dispersant water=1.330, Absorption=1.0 and Obscuration=7-13%. The sample suspension is prepared by mixing approximately 5 mg of sample with 10 ml of water adding 2 drops of Tween 80 in a 25 ml beeker. The Dispersion Unit (Malvern instruments) is filled with water and the pump/stirrer in the dispersion unit tank is turned to 3500 rpm and then down to zero to clear any bubbles. The sample suspension is sonicated for 1 minute. The pump/stirrer is turned to 1000 rpm and then the background is measured. Slowly, the prepared suspension sample is dropped into the dispersion unit until a stabilized obscuration of 7-13% is reached, and the analysis started. The analysis was done in triplicate.

According to the present invention the material of the capsules in which is filled the pharmaceutical composition of the present invention is selected from the list comprising, but not limited to, hard gelatin, HPMC, plant-based material, fish gelatin, starch, pullulan, polyvinl acetate (PVA), and soft gelatin. Preferably the capsules are made of HPMC capsules or hard gelatine, or plant-based material.

According to the present invention the capsules in which is filled the pharmaceutical composition of the present invention have a range of sizes comprised between 000 and 5, preferably comprised between 00el and 4, even more preferably comprised between 00 and 3. Even more preferably the capsules has size 2 or 3. Depending on the chosen inhaler and the required dosage, the skilled person in the art shall select the most suitable size. According to a preferred embodiment of the invention, when RS01 Plastiape device is used, the size of the capsules would be 2 or 3.

According to the present invention, the composition shows an uniformity of distribution of the compound of formula (I), expressed as coefficient of variation (CV) also known as relative standard deviation (RSD), which is less than 5.0%, preferably equal to or less than 2.5%, as shown in Table 1 of Example 2 in the experimental part.

Furthermore the compositions is physically and chemically stable upon storage into the inhaler at room temperature at 60% relative humidity for at least 24 months.

The present invention is also directed to a process for preparing the compositions disclosed herein comprising the step of mixing the fraction of fine particles a), the fraction of coarse particles b) with both the micronized active ingredients.

The carrier particles comprising the fraction of fine particles and the fraction of coarse particles may be prepared by mixing in suitable apparatus known to the skilled person, for example a Turbula™ mixer. The two fractions are preferably mixed in a Turbula™ mixer operating at a rotation speed of 16 r.p.m. for a period comprised between 30 and 300 minutes, preferably between 150 and 240 minutes.

The mixture of the carrier particles with the active ingredient particles may be carried out by mixing the components in suitable apparatus known to the skilled person, such as Turbula™ mixer for a period sufficient to achieve the homogeneity of the active ingredient in the final mixture, preferably comprised between 30 and 120 minutes, more preferably between 45 and 100 minutes.

Optionally, in an alternative embodiment, one active ingredient is first mixed with a portion of the carrier particles and the resulting blend is forced through a sieve, then, the further active ingredient and the remaining part of the carrier particles are blended with the sieved mixture; and finally the resulting mixture is sieved through a sieve, and mixed again.

The skilled person shall select the mesh size of the sieve depending on the particle size of the coarse particles.

In another preferred embodiment, the present invention provides the drug product of the invention, for use for the treatment of an inflammatory or obstructive respiratory disease. As an alternative, the invention provides the pharmaceutical composition according to the invention, upon administration by the single-dose inhaler according to the invention for use for the treatment of an inflammatory or obstructive respiratory disease.

In a further preferred embodiment, the present invention provides the drug product as defined above, for use for the treatment of an inflammatory or obstructive respiratory disease selected from: asthma, chronic obstructive pulmonary disease (COPD), bronchiectasis, chronic bronchitis, pneumonia, acute respiratory distress syndrome (ARDS), pulmonary emphysema, smoking-induced emphysema and cystic fibrosis.

Although the carrier shall consist of the particles according to the invention, the composition may comprise further active ingredients and, optionally other excipients, for example sweeteners and flavoring agents.

The further active ingredients could be selected from those currently utilized for the prevention and treatment of respiratory diseases by inhalation, for example beta₂-agonists, corticosteroids and anticholinergic agents.

In an even further preferred embodiment, the present invention provides the drug product of the invention as defined above as add-on to a single, double or triple therapy.

In another preferred embodiment, the present invention provides the drug product of the invention as defined above wherein the single, double or triple therapy active agents are selected from beta₂-agonists, corticosteroids and anticholinergic agents.

In a preferred embodiment, the present invention is directed to a drug product comprising a single-dose dry powder inhalation device, comprising an inhaler body (2) defining a recess (3) for a capsule (4), wherein the capsule (4) holds herein a pharmaceutical composition to be inhaled, a nosepiece or mouthpiece (5) communicating with the recess (3), at least one rupturing element (7) coupled to the inhaler body (2) and configured for rupturing the capsule (4) to allow an outside airflow to be mixed with the pharmaceutical composition of the capsule (4) and inhaled through the nosepiece or the mouthpiece (5), and a pharmaceutical composition filled in a capsule, the pharmaceutical composition comprising micronized particles having a size comprised between 0.1 and 15 micron of a compound of formula (I) and carrier particles, wherein the inspiratory flow rate of said inhalation device is equal to lower than 65 l/min, wherein the nominal dose of the compound of formula (I) per actuation is comprised between 450 and 600 μg, and wherein the carrier is constituted of:
a) a fraction of fine particles made of a mixture composed of 98 percent by weight of particles of alpha-lactose monohydrate and 2 percent by weight of magnesium stearate, said mixture having a mass median diameter equal to or lower than 6 micron; b) a fraction of coarse particles constituted of alpha-lactose monohydrate having a mass diameter comprised between 210 and 355 micron and the ratio between the fine particles and the coarse particles being 10:90 percent by weight.

In an even more preferred embodiment, the particle size of the compound of formula (I) has the dv(0.1) comprised between 0.5 and 1 micron, the dv(0.5) comprised between 1.9 and 2.5 micron, the dv(0.9) comprised between 4 and 6 micron.

In another preferred embodiment, the invention is directed to a pharmaceutical composition according to the invention for use for the treatment of a respiratory disease, wherein said composition is administered using a single-dose dry powder inhalation device whose inspiratory flow rate is comprised between 30 l/min and 65 l/min.

In an even further preferred embodiment, the invention provides a method for the treatment of a respiratory disease, wherein the method comprises administering the compound of formula (I) by inhalation to a patient, wherein the drug product is as described according to the invention and wherein the nominal dose of the compound of formula (I) per actuation is comprised between 450 and 600 μg.

In another embodiment, the invention provides a process for the preparation of a pharmaceutical composition according to the invention, said process comprising the step of:
a) preparing microparticles constituted of a mixture composed of particles made of physiologically acceptable pharmacologically-inert material and particles of the additive, the inert material and the additive being first-mixed together and then co-micronized;
b) mixing the microparticles of step a) with coarse particles of a physiologically acceptable pharmacologically-inert material such that microparticles adhere to the surface of the coarse particles;
c) adding by mixing the active particles in the micronized form to the particles of step b).

In an even further preferred embodiment, the invention provides a process for manufacturing a drug product comprising a step of filling the medicament chamber of a single dry powder inhalation device with a capsule filled with a pharmaceutical composition according to the invention.

### EXPERIMENTAL PART

### Abbreviations

Moc=Micro-Orifice Collector; IP=Induction Port; PS=Pre Separator

### Example 1: Preparation of the Composition of the Invention

The composition of the invention was prepared according to the procedure disclosed in WO 2012/016889.

### Example 2: Content of the Compound of Formula (I) (μg)/20 mg of the Composition of the Invention, ±St.Dev and CV % (n=6)

The uniformity of drug content in the blends was determined with HPLC. The analysis was conducted on 6 samples, collected randomly in the mixture, dissolved in 100 ml of acetonitrile/water (60/40) v/v used as solvent. 20 mg were weighed for each sample.

TABLE 1
content of the compound of formula (I) (μg)/20 mg of the composition
of the invention, ±St. Dev and CV % (n = 6)

| | 400 μg of compound of | 800 μg of compound of |
|---|---|---|
| Samples | formula (I)/20 mg | formula (I)/20 mg |
| #1 | 416.8 | 799.1 |
| #2 | 413.1 | 802.7 |
| #3 | 410.5 | 824.3 |
| #4 | 397.9 | 791.1 |
| #5 | 410.8 | 797.6 |
| #6 | 382.9 | 826.1 |
| Mean (±St. Dev) | 405.3 ± 12.7 | 806.8 ± 14.7 |
| CV (%) | 3.1 | 1.8 |

The blends in Table 1 show an excellent accuracy and uniformity of distribution (precision as CV) of the active ingredient.

### Example 3: Determination of the Aerodynamic Particle Size Distribution (APSD)

The in vitro aerodynamic assessment was carried out using a Next Generation Impactor (NGI), following the procedure detailed in the European Pharmacopoeia 10.0 in the 2.9.18 “Preparation for inhalation: Aerodynamic assessment of fine particles” chapter at pp. 347-360.

Nexthaler (Chiesi, Parma, Italy), combined with the composition prepared according to WO 2012/016889 at 400 μg and 800 μg was considered as the Reference Products.

RS01 high resistance with code 239700002AA and RS01 ultra-high resistance with code 239700005AA devices (Plastiape, Osnago, LC Italy) were used to conduct the analysis with the Drug Products according to the composition invention at 400 μg and 800 μg. The capsules used were Quali-V®-I, size 3 TAA TAA (Qualicaps Europe, S.A.U.) and loaded with about 20 mgs. The DPI inhalers were activated at a pressure drop of 4 kPa corresponding at a flow rate of 57.5 L/min for Nexthaler of 65 L/min for HR RS01 and 40 L/min for UHR RS01, for a duration of time sufficient to sample an air volume of 4.0 liters. The NGI was connected to the vacuum pump and the airflow was fixed using a flowmeter. The analysis was performed under critical flow control conditions. The device was connected to the NGI through a rubber adaptor, and one single dose was discharged and collected into the apparatus. Three different devices for each type of DPI were used. The drug remaining in capsule and device (only for RS01 analysis), and the drug deposited in the different portions of the impactor was recovered using acetonitrile/water (60/40) v/v as solvent. The samples were filtered with RC filter (0.45 μm) and quantified using HPLC to determine the amount of drug.

The metered dose (MD) was calculated by summing the drug recovered from the impactor (IP, PS, stages 1 to 7 and MOC) and the drug remaining in the inhaler (capsule and device). It wasn't possible to quantify MD for the multidose Nexthaler DPI since it is a reservoir multidose inhaler and cannot be wet and rinsed at the end of the experiment.

The Emitted Dose (ED) is the amount of drug leaving the device and entering the impactor and was calculated by summing the drug recovered from the impactor (IP, PS, stages 1 to 7 and MOC).

The drug deposition in the impactor allowed the calculation of the aerodynamic parameters.

The mass median aerodynamic diameter (MMAD) and Geometric Standard Deviation (GSD) were determined by plotting the cumulative percentage of mass less than the stated aerodynamic diameter for each NGI stage on a probability scale versus the aerodynamic diameter of the stage on a logarithmic scale. Linear regression of the six data points closest to 50% of the cumulative particle mass that entered the impactor was performed to compute the MMAD and GSD.

The Fine Particle Mass (FPM) was calculated as the mass of drug <5 μm (calculated from the log-probability plot equation) and the Fine Particle Fraction (FPF) was determined as the ratio between FPD and ED in percent.

The Extra Fine Particle Mass (EFPM) was calculated as the mass of drug below 2 μm (calculated from the log-probability plot equation) and the Extra Fine Particle Fraction (EFPF) was determined as the ratio between EFPD and ED in percent.

TABLE 2
APSD of Nexthaler device loaded with
the Reference Composition at 400 μg

| Reference | | | | | |
|---|---|---|---|---|---|
| Composition | Device | Device | Device | Mean ± St. | |
| (400 μg/20 mg) | #1 | #2 | #3 | Dev | CV(%) |
| Shot weight (mg) | 19.6 | 21.4 | 22.1 | 21.0 ± 1.3 | 6.1 |
| Emitted dose (μg) | 341.3 | 384.3 | 403.9 | 376.5 ± 32.1 | 8.1 |
| MMAD (μm) | 2.12 | 2.04 | 2.07 | 2.08 ± 0.04 | 1.95 |
| GSD | 2.47 | 2.55 | 2.50 | 2.51 ± 0.04 | 1.60 |
| Fine Particle | 197.4 | 238.6 | 253.3 | 229.8 ± 29.0 | 12.6 |
| Mass (μg) | | | | | |
| Fine Particle | 57.8 | 62.1 | 62.7 | 60.9 ± 2.6 | 4.3 |
| Fraction (%) | | | | | |
| Extra Fine Particle | 108.2 | 135.2 | 140.9 | 128.1 ± 17.5 | 13.6 |
| Mass (μg) | | | | | |
| Extra Fine Particle | 31.7 | 35.2 | 34.9 | 33.9 ± 1.9 | 5.7 |
| Fraction (%) | | | | | |

Shot weight (mg), Emitted Dose (μg), MMAD (μm), GSD, FPM (μg), FPF (%), EFPM (μg), EFPF (%) mean values of Nexthaler device loaded with the Reference Composition (400 μg of compound of formula (I)/20 mg) ± St. dev and CV % in (n = 3)

TABLE 3
APSD of Nexthaler device loaded with
the Reference Composition at 800 μg

| Reference | | | | | |
|---|---|---|---|---|---|
| Composition | Device | Device | Device | Mean ± St. | |
| (800 μg/20 mg) | #1 | #2 | #3 | Dev | CV(%) |
| Shot weight (mg) | 20.7 | 21.8 | 20.0 | 20.8 ± 0.9 | 4.4 |
| Emitted dose (μg) | 823.3 | 859.4 | 769.8 | 817.5 ± 45.1 | 5.5 |
| MMAD (μm) | 2.00 | 2.00 | 2.05 | 2.02 ± 0.05 | 1.43 |
| GSD | 2.42 | 2.49 | 2.40 | 2.44 ± 0.05 | 1.92 |
| Fine Particle | 417.8 | 467.2 | 394.7 | 426.6 ± 37.0 | 8.7 |
| Mass (μg) | | | | | |
| Fine Particle | 50.8 | 54.4 | 51.3 | 52.1 ± 2.0 | 3.7 |
| Fraction (%) | | | | | |
| Extra Fine Particle | 234.8 | 265.3 | 217.8 | 239.3 ± 24.0 | 10.0 |
| Mass (μg) | | | | | |
| Extra Fine Particle | 28.5 | 30.9 | 28.3 | 29.2 ± 1.4 | 4.9 |
| Fraction (%) | | | | | |

Shot weight (mg), Emitted Dose (μg), MMAD (μm), GSD, FPM (μg), FPF (%), EFPM (μg), EFPF (%) mean values of Nexthaler device loaded with the Reference Composition (800 μg of compound of formula (I)/20 mg) ± St. dev and CV % in (n = 3)

TABLE 4
APSD of HR RS01 device loaded with the composition
of the invention at 400 μg (not-working Example)

| Composition of | | | | | |
|---|---|---|---|---|---|
| the invention | Device | Device | Device | Mean ± St. | |
| (400 μg/20 mg) | #1 | #2 | #3 | Dev | CV(%) |
| Shot weight (mg) | 19.9 | 20.0 | 19.9 | 19.9 ± 0.1 | 0.3 |
| Metered dose (μg) | 372.5 | 380.3 | 391.4 | 381.4. ± 9.5 | 2.5 |
| Emitted dose (μg) | 291.3 | 296.0 | 286.8 | 291.3 ± 4.6 | 1.6 |
| MMAD (μm) | 1.71 | 1.71 | 1.64 | 1.69 ± 0.04 | 2.40 |
| GSD | 2.64 | 2.73 | 2.59 | 2.65 ± 0.07 | 2.60 |
| Fine Particle | 217.6 | 227.8 | 225.1 | 223.5 ± 5.3 | 2.4 |
| Mass (μg) | | | | | |
| Fine Particle | 74.7 | 77.0 | 78.5 | 76.7 ± 1.9 | 2.5 |
| Fraction (%) | | | | | |
| Extra Fine | 135.5 | 142.2 | 142.2 | 140.0 ± 3.9 | 2.8 |
| Particle Mass (μg) | | | | | |
| Extra Fine | 46.5 | 48.0 | 49.6 | 48.1 ± 1.5 | 3.2 |
| Particle | | | | | |
| Fraction (%) | | | | | |

Shot weight (mg), Metered dose (μg), Emitted Dose (μg), MMAD (μm), GSD, FPM (μg), FPF (%), EFPM (μg), EFPF (%) mean values of RS01 device loaded with the composition of the invention (400 μg of compound of formula (I)/20 mg) ± St. dev and CV % in (n = 3)

TABLE 5
APSD of HR RS01 device loaded with the
composition of the invention at 800 μg

| Composition of | | | | | |
|---|---|---|---|---|---|
| the invention | Device | Device | Device | Mean ± St. | |
| (800 μg/20 mg) | #1 | #2 | #3 | Dev | CV(%) |
| Shot weight (mg) | 19.2 | 19.4 | 18.9 | 19.2 ± 0.2 | 1.2 |
| Metered dose (μg) | 817.3 | 838.4 | 819.0 | 824.9 ± 11.7 | 1.4 |
| Emitted dose (μg) | 627.1 | 624.8 | 639.0 | 630.3 ± 7.6 | 1.2 |
| MMAD (μm) | 1.61 | 1.74 | 1.70 | 1.68 ± 0.07 | 3.96 |
| GSD | 2.62 | 2.41 | 2.47 | 2.50 ± 0.11 | 4.30 |
| Fine Particle | 461.5 | 515.1 | 531.9 | 502.8 ± 36.8 | 7.3 |
| Mass (μg) | | | | | |
| Fine Particle | 73.6 | 82.4 | 83.2 | 79.8 ± 5.4 | 6.7 |
| Fraction (%) | | | | | |
| Extra Fine | 294.8 | 311.9 | 327.8 | 311.5 ± 16.5 | 5.3 |
| Particle Mass (μg) | | | | | |
| Extra Fine | 47.0 | 49.9 | 51.3 | 49.4 ± 2.2 | 4.4 |
| Particle | | | | | |
| Fraction (%) | | | | | |

Shot weight (mg), Metered dose (μg), Emitted Dose (μg), MMAD (μm), GSD, FPM (μg), FPF (%), EFPM (μg), EFPF (%) mean values of RS01 device loaded with the composition of the invention (800 μg of CHF001/20 mg) ± St. dev and CV % in (n = 3)

TABLE 6
APSD of UHR RS01 device loaded with the composition
of the invention at 400 μg (not working Example)

| Composition of | | | | | |
|---|---|---|---|---|---|
| the invention | Device | Device | Device | Mean ± St. | |
| (400 μg/20 mg) | #1 | #2 | #3 | Dev | CV(%) |
| Shot weight (mg) | 19.9 | 20.1 | 20.1 | 20.0 ± 0.1 | 0.6 |
| Metered dose (μg) | 453.5 | 478.6 | 459.4 | 463.9 ± 13.1 | 2.8 |
| Emitted dose (μg) | 278.3 | 299.1 | 284.1 | 287.2 ± 10.7 | 3.7 |
| MMAD (μm) | 1.68 | 1.73 | 1.69 | 1.70 ± 0.03 | 1.56 |
| GSD | 2.47 | 2.27 | 2.43 | 2.39 ± 0.10 | 4.38 |
| Fine Particle | 209.3 | 229.8 | 215.0 | 218.0 ± 10.6 | 4.9 |
| Mass (μg) | | | | | |
| Fine Particle | 75.2 | 76.8 | 75.7 | 75.9 ± 0.8 | 1.1 |
| Fraction (%) | | | | | |
| Extra Fine | 131.3 | 141.3 | 134.4 | 135.7 ± 5.1 | 3.8 |
| Particle Mass (μg) | | | | | |
| Extra Fine | 47.2 | 47.2 | 47.3 | 47.2 ± 0.1 | 0.2 |
| Particle | | | | | |
| Fraction (%) | | | | | |

Shot weight (mg), Metered dose (μg), Emitted Dose (μg), MMAD (μm), GSD, FPM (μg), FPF (%), EFPM (μg), EFPF (%) mean values of RS01 device loaded with the composition of the invention (400 μg of compound of formula (I)/20 mg) ± St. dev and CV % in (n = 3)

TABLE 7
APSD of UHR RS01 device loaded with the
composition of the invention at 800 μg

| Composition of | | | | | |
|---|---|---|---|---|---|
| the invention | Device | Device | Device | Mean ± St. | |
| (800 μg/20 mg) | #1 | #2 | #3 | Dev | CV(%) |
| Shot weight (mg) | 20.3 | 20.2 | 20.2 | 20.2 ± 0.1 | 0.3 |
| Metered dose (μg) | 906.0 | 774.0 | 804.3 | 828.1. ± 69.1 | 8.3 |
| Emitted dose (μg) | 621.7 | 559.6 | 558.6 | 580.0 ± 36.2 | 6.2 |
| MMAD (μm) | 1.86 | 1.81 | 1.78 | 1.82 ± 0.04 | 2.22 |
| GSD | 2.17 | 2.12 | 2.15 | 2.14 ± 0.02 | 1.10 |
| Fine Particle | 450.7 | 425.5 | 414.6 | 430.3 ± 18.5 | 4.3 |
| Mass (μg) | | | | | |
| Fine Particle | 72.5 | 76.0 | 74.2 | 74.3 ± 1.8 | 2.4 |
| Fraction (%) | | | | | |
| Extra Fine | 259.9 | 245.4 | 251.8 | 252.4 ± 7.3 | 2.9 |
| Particle Mass (μg) | | | | | |
| Extra Fine | 41.8 | 43.9 | 45.1 | 43.6 ± 1.6 | 3.8 |
| Particle | | | | | |
| Fraction (%) | | | | | |

Shot weight (mg), Metered dose (μg), Emitted Dose (μg), MMAD (μm), GSD, FPM (μg), FPF (%), EFPM (μg), EFPF (%) mean values of RS01 device loaded with the composition of the invention (800 μg of compound of formula (I)/20 mg) ± St. dev and CV % in (n = 3)

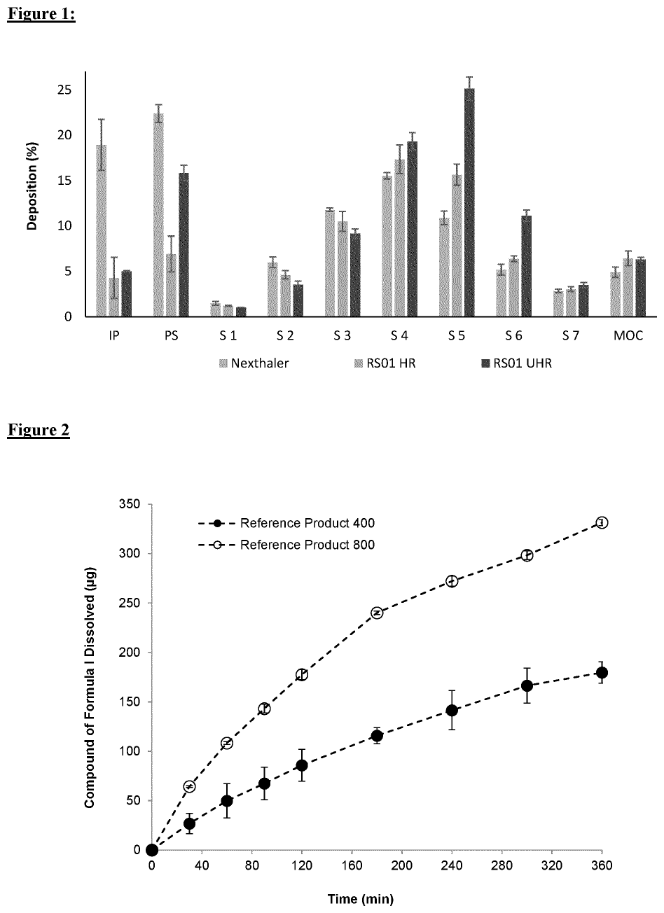

As it can be seen from FIG. 1, the aerosolization of the composition of the invention from capsule inhaler (800 μg) leads to a significantly lower deposition of the drug in the preseparator (PS) and in IP, both simulating the oropharyngeal tract. In parallel, the deposition in the stages that collect the fine particle fraction of drug (S4, S5, S6 and MOC) is higher using the capsule inhaler, both for HR or UHR RS01 device. Consequently, the dose loaded into the inhaler can be decreased to match the fine particle fractions emitted by Nexthaler.

### Example 4: Dissolution Test

In vitro dissolution tests were carried out to compare the performance of the Reference Products and the drug product of the invention at 800 μg, using RespiCell™ (EU registration No 006649570-0001), a vertical diffusion cell apparatus that comprises a 170 cm³ reservoir filled with the dissolution media and a side arm of 10 cm length. The apparatus is composed of an upper part, the donor chamber, and a lower part, the receptor chamber, linked by a clamp and separated by a glass fiber filter, used as diffusion membrane, and sit horizontally in contact with the dissolution medium. The receptor chamber contains a magnetic stirrer inside it. Type A/E glass fiber filter of 76 mm diameter (PALL Corporation, Port Washington; NY, USA) were employed as diffusion membrane. The dissolution medium employed for the analysis was phosphate-buffer saline (PBS) with 0.5% of Sodium dodecyl sulfate (SDS). RespiCell™ was connected to a heating thermostat (Lauda eco silver E4, DE) set at 37±0.5° C. The receptor chamber was filled with the dissolution medium and sampled at preset time interval through the side arm of the cell. 1 ml of dissolution medium were applied on the filter to get it completely wet before the analysis. The analysis was conducted by employing the fine fraction deposited on the filter after aerosolization by Fast Screening Impactor (FSI). The in vitro aerodynamic assessment was carried following the procedure detailed in the the procedure detailed in the European Pharmacopoeia 10.0 in the 2.9.18 “Preparation for inhalation: Aerodynamic assessment of fine particles” chapter at p 347-360. HR RS01 and UHR RS01 devices were used to conduct the analysis. The capsules used were Quali-V®-I, size 3 TAA TAA (Qualicaps Europe, S.A.U.) and loaded with about 20 mg. The HR RS01 inhaler was activated at a pressure drop of 4 kPa corresponding at a flow rate of 65 L/min for a duration of time sufficient to sample an air volume of 4.0 liters. The UHR RS01 inhaler was activated at a pressure drop of 4 kPa corresponding at a flow rate of 40 L/min for a duration of time sufficient to sample an air volume of 4.0 liters. The FSI was connected to the vacuum pump and the airflow was fixed using a flow meter. The analysis was performed under critical flow control conditions. The device was connected to the FSI through a rubber adaptor; two capsules were aerosolized, and two doses were collected into the apparatus. The analysis was done in triplicate for each selected composition. After aerosolization, the filter was removed by the FSI and located on the RespiCell™, between the donor chamber and the receptor chamber. 1 mL of the receiving solution was removed at fixed intervals by the receptor chamber and replaced with 1 mL of fresh dissolution medium after every withdrawal to maintain a constant volume. In order to assess the amount of drug not dissolved or entrapped in the filter, the residual not-dissolved powder was recovered by washing the filter with 10 mL of acetonitrile:water 60:40, at the end of experiment. The amount of drug in the samples was assessed by HPLC. The data were expressed as percentage of the compound of formula (I) dissolved and 100% of the dissolution corresponded to the drug amount dissolved at the end of experiment. The dissolution profiles were examined in terms of fraction and overall amount dissolved over time, using the difference (f1) and similarity factors (f2). The difference factor (f1) calculates the percent difference between two dissolution profiles at each time point and is a measurement of the relative error between the two profiles. The difference factor (f1) is calculated as follows:

$${f}_{1} = ( \frac{{\sum }_{t = 1}^{n} \vert {R}_{t} - {T}_{t} \vert}{{\sum }_{t = 1}^{n} {R}_{t}} ) \times 100$$

The similarity factor (f2) is calculated as follows:

$${f}_{2} = 50 \times log [ \frac{100}{\sqrt{1 + \frac{{\sum }_{t = 1}^{n} {( {R}_{t} - {T}_{t} )}^{2}}{n}}} ]$$

n is the number of time points, Rt is the mean dissolution value for the reference product at time t, and Tt is the mean dissolution value for the test product at that same time point. The evaluation of f1 and f2 is based on the following conditions: a minimum of three time points (zero excluded) should be considered, and the time points should be the same for the two compositions, and not more than one mean value should exceed 85% of the dissolved drug for any of the compositions. In addition, the relative standard deviation (coefficient of variation) should be less than 20% for the first time point and less than 10% for the other time points considered. A difference factor (f1) value lower than 15 (0-15) indicates no significant difference between the dissolution profiles. A similarity factor (f2) value higher than 50 (50-100) indicates similarity between the two dissolution profiles.

To test the reliability of the method, the in vitro dissolution profiles of the two Reference Products at 400 and 800 μg dose were provided. The results reported in FIG. 2 show an excellent proportionality between the dose and the dissolved amount of drug.

Then, the comparative in vitro dissolution tests between the Reference Product and the drug product of the invention were performed, and as it can be appreciated from FIG. 3, showed a lower dissolution profile for the Reference Product released by Nexthaler compared to the drug product of the invention released by HR RS01 and UHR RS01 device.

Based on these in vitro results and the indications/assumptions of the inhalation Bioclassification System (Hastedt J E et al AAPS/FDA/USP Workshop March 16-17^{th}, Baltimore, AAPS Open, 2016, 2 (1), 2016), an IVIV correlation model could be set up to demonstrate plausible bioequivalence, and candidates the drug product of the invention as a biowaiver.

1. A drug product, comprising a single-dose dry powder inhalation device, comprising an inhaler body (2) defining a recess (3) for a capsule (4), wherein the capsule (4) holds herein a pharmaceutical composition to be inhaled, a nosepiece or mouthpiece (5) communicating with the recess (3), at least one rupturing element (7) coupled to the inhaler body (2) and configured for rupturing the capsule (4) to allow an outside airflow to be mixed with the pharmaceutical composition of the capsule (4) and inhaled through the nosepiece or the mouthpiece (5), and
a pharmaceutical composition filled in a capsule, the pharmaceutical composition comprising micronized particles having a size of from 0.1 to 15 microns of a compound of formula (I) 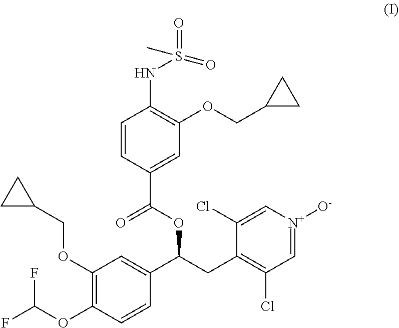 [SMILES: CS(=O)(=O)Nc1ccc(C(=O)O[C@@H](Cc2c(Cl)c[n+]([O-])cc2Cl)c2ccc(OC(F)F)c(OCC3CC3)c2)cc1OCC1CC1]
and carrier particles,
wherein the inspiratory flow rate of said inhaler is from 30 l/min to 65 l/min at 4 kPa of pressure drop, and
wherein the unitary nominal dose of the compound of formula (I) is from 450 to 600 μg.

2. The drug product according to claim 1, wherein the unitary nominal dose is from 480 to 550 μg.

3. The drug product according to claim 1, wherein the carrier is selected from the group consisting of a polyol, a crystalline sugar, an inorganic salt, an organic salt, an organic compound, a polysaccharide, and an oligosaccharide.

4. The drug product according to claim 1, wherein the carrier particles comprise a mixture of particles having a size of one or a few hundred microns, and particles having a size up to a few tens of microns, and a ternary agent.

5. The drug product according to claim 1, wherein the carrier comprises:
a) a fraction of fine particles made of a mixture comprising 90 to 99.5 percent by weight of particles of a physiologically acceptable excipient and 0.5 to 10 percent by weight of a ternary agent, said mixture having a volume median diameter lower than 20 microns; and
b) a fraction of coarse particles comprising a physiologically acceptable excipient having a volume median diameter equal to or higher than 100 microns as measured by means of laser diffraction or sieve analyzer,
wherein the ratio between the fine particles and the coarse particles is comprised between 1:99 and 30:70 percent by weight.

6. The drug product according to claim 1, wherein the inspiratory flow rate is from 40 l/min to 65 l/min at 4 kPa of pressure drop.

7. A method for the treatment of a respiratory disease, comprising administering to a subject in need thereof an effective amount of a pharmaceutical composition comprising micronized particles having a size of from 0.1 to 15 microns of a compound of formula (I) 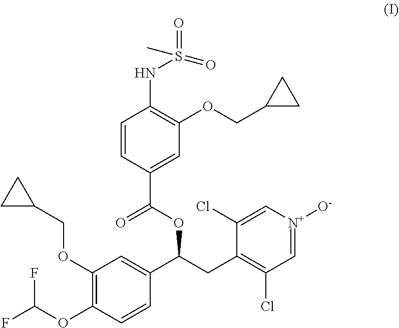 [SMILES: CS(=O)(=O)Nc1ccc(C(=O)O[C@@H](Cc2c(Cl)c[n+]([O-])cc2Cl)c2ccc(OC(F)F)c(OCC3CC3)c2)cc1OCC1CC1]
and carrier particles,
wherein said composition is administered using a single-dose dry powder inhalation device whose inspiratory flow rate is from 30 l/min to 65 l/min, and
wherein the unitary dose of the compound of formula (I) is from 450 to 600 μg.

8. The method according to claim 7, wherein the respiratory disease is asthma or chronic obstructive pulmonary disease.

9. A process for the preparation of a drug product according to claim 1, said process comprising:
a) preparing microparticles comprising a mixture comprising particles made of physiologically acceptable pharmacologically-inert material and particles of an additive, the inert material and the additive being first-mixed together and then co-micronized;
b) mixing the microparticles obtained in step a) with coarse particles of a physiologically acceptable pharmacologically-inert material such that microparticles adhere to the surface of the coarse particles;
c) adding by mixing the active particles in the micronized form to the particles obtained in step b) to obtain the final pharmaceutical composition;
d) filing the obtained final pharmaceutical composition in a capsule; and
c) loading the capsule in a medicament chamber of a single dry powder inhalation device.
